(19) United States
(12) Patent Application Publication
(10) Pub. No.: US 2017/0065568 A1
(43) Pub. Date: Mar. 9, 2017

(54) METHODS AND COMPOUNDS FOR CALCIUM ION CHANNEL REGULATION

(71) Applicant: Duke University, Durham, NC (US)

(72) Inventors: Geoffrey S. Pitt, Durham, NC (US); Adam S. Barnett, Durham, NC (US); Xinhe Yin, Cary, NC (US)

(21) Appl. No.: 15/154,140

(22) Filed: May 13, 2016

Related U.S. Application Data
(63) Continuation of application No. 14/209,345, filed on Mar. 13, 2014, now abandoned.
(60) Provisional application No. 61/792,992, filed on Mar. 15, 2013.

(51) Int. Cl. A61K 31/4422; A61K 31/40; A61K 45/06; A61K 47/48

(52) U.S. Cl. CPC A61K 31/4422; A61K 47/48315; A61K 31/40; A61K 47/48023; A61K 47/48015; A61K 45/06

(57) ABSTRACT

The present invention generally relates to methods of modulating Caᵥ1.2 channels and Caᵥ1.2 channel activators.

### STATEMENT OF PRIORITY

This application is a continuation application of, and claims priority to, U.S. application Ser. No. 14/209,345 filed Mar. 13, 2014, which claims the benefit, under 35 U.S.C. §119(e), of U.S. Provisional Application Ser. No. 61/792,992, filed Mar. 15, 2013, the entire contents of which are incorporated by reference herein.

### FIELD OF THE INVENTION

The present invention generally relates to methods of modulating Caᵥ1.2 channels and Caᵥ1.2 channel activators.

### BACKGROUND

Osteoporosis is a growing major public health issue for which treatment options are limited. It is an object of the present disclosure to provide new methods and therapeutic agents for treating and/or preventing disorders such as, but not limited to, osteoporosis.

### SUMMARY OF EMBODIMENTS

Embodiments of the present invention are directed to a method of increasing bone mass in a subject, comprising modulating Caᵥ1.2 channel activity in bone of the subject, thereby increasing bone mass in the subject.

Some embodiments of the present invention are directed to a method of preventing and/or treating osteoporosis in a subject in need thereof, comprising modulating Caᵥ1.2 channel activity in bone of the subject, thereby preventing and/or treating osteoporosis in the subject.

Further embodiments are of the present invention are directed to a method of preventing or slowing bone mineral density loss in bone of a subject in need thereof, comprising modulating Caᵥ1.2 channel activity in bone of the subject, thereby preventing or slowing bone mineral density loss in bone of the subject.

Some embodiments of the present invention are directed to a method of preventing the occurrence of compression fractures due to osteoporosis, comprising modulating Caᵥ1.2 channel activity in bone of the subject, thereby preventing said occurrence of compression fractures due to osteoporosis.

Certain embodiments of the present invention are directed to Caᵥ1.2 channel activators. A Caᵥ1.2 channel activator may mimic a gain-of-function mutant L-type (Cᵥ1.2) voltage-gated Ca²⁺ channel. In some embodiments, a Caᵥ1.2 channel activator may be specific for bone and/or may target bone.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### BRIEF DESCRIPTION OF THE DRAWINGS

FIG. 1 shows, the increased bone mass in Prxl-Caᵥ1.2^{TS}mice. A) Radiographs from Prx⁺ and Prx⁻ littermates, age 5 weeks. Arrows pointing to radius/ulna or tibia/fibula show increased density in the appendicular skeleton. B) μCT of femur (mid-shaft) showing increased cortical bone obscuring the marrow cavity. C) Safranin O staining of proximal femur; bracket indicates zone of proliferating and hypertrophic chondrocytes. Cartilage stains dark grey; cytoplasm light grey; and nuclei black.

FIG. 2 shows activation of the Caᵥ1.2^{TS} channel in adult animals. The gain-of-function channel was activated in adults (8 week old mice) by a promoter that turns on the channel in all bones. Radiographs were obtained at 5 months of age. Tail vertebrae are denser when the gain-of-function channels are activated.

### DETAILED DESCRIPTION

The present invention will now be described more fully hereinafter. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety for the portions relevant to the sentence and/or paragraph in which the reference is present. In the event of conflicting terminology; the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” as used herein should not be interpreted as equivalent to “comprising.”

The term “about” as used herein when referring to a measurable value such as an amount or concentration (e.g., the amount of a substrate), is meant to encompass variations of ±10%, ±5%, ±1%, ±0.5%, or even ±0.1% of the specified measurable value as well as the specified value. For example, “about X” where X is the measurable value, is meant to include X as well as variations of ±10%, ±5%, ±1%, ±0.5%, or even ±0.1% of X. A range provided herein for a measureable value may include any other range and/or individual value therein.

According to some embodiments, provided is a method of modulating a Caᵥ1.2 channel activity in bone of a subject. A “Caᵥ1.2 channel” is an L-type, voltage-gated, calcium channel that is encoded by the calcium channel, voltage-dependent, L type, alpha 1C subunit (CACNA1C) gene. A Caᵥ1.2 channel may transport calcium ions (e.g., Ca²⁺) into the cell in which the Caᵥ1.2 channel is present and may aid in the cell's ability to generate and transmit electrical signals. A Caᵥ1.2 channel may regulate a cellular response and/or process, such as, but not limited to neuronal activity, muscle contraction, and hormone release. In some embodiments, a method of the present invention modulates an endogenous Caᵥ1.2 channel in a bone of a subject.

“Modulate,” “modulating,” “modulation,” and grammatical variations thereof as used herein refer to an increase or a reduction in a Caᵥ1.2 channel activity in bone of a subject compared to the activity of the Caᵥ1.2 channel in the bone in the absence of a method of the present invention. As used herein, the terms “increase,” “increases,” “increased,” “increasing” and similar terms (e.g., activation, upregulation, and the like) indicate an elevation in activity of at least about 5%, 10%, 25%, 50%, 75%, 100%, 150%, 200%, 300%, 400%, 500%, or more compared to the activity in the absence of a method of the present invention. As used herein, the terms “reduce,” “reduces,” “reduced,” “reduction” and similar terms (e.g., inhibition, downregulation, and the like) refer to a decrease in activity of at least about 5%, 10%, 25%, 35%, 50%, 75%, 80%, 85%, 90%, 95%, 97%, or more compared to the activity in the absence of a method of the present invention. In some embodiments, a method of modulating a Caᵥ1.2 channel activity may be carried out by an osteoblast-specific mechanism and/or a chondrocyte-specific mechanism.

Any activity in which a Caᵥ1.2 channel plays a direct and/or indirect role may be modulated by a method of the present invention. In certain embodiments, a method of the present invention increases Caᵥ1.2 channel activity in bone of a subject. For example, one or more functions of a Cᵥ1.2 channel (e.g., transportation of calcium ions into the cell, generation and/or transmission of electrical signals, regulation of a cellular response and/or process, etc.) may be increased compared to the function of the Caᵥ1.2 channel in the absence of a method of the present invention. The increase in Caᵥ1.2 channel activity may be an overall increase in function of the Caᵥ1.2 channel (e.g., the cumulative amount of activity of the Caᵥ1.2 channel may increase compared to the cumulative amount of activity of the Caᵥ1.2 channel in the absence of a method of the present invention).

In some embodiments, a method of the present invention increases the transport of calcium ions (e.g., Ca²⁺) into the cell in which the Caᵥ1.2 channel is present. A method of the present invention may increase the concentration of cytoplasmic calcium ions in the cell in which the Caᵥ1.2 channel is present compared to the concentration of cytoplasmic calcium ions in the cell or a comparable cell in the absence of a method of the present invention. A method of the present invention may increase the concentration of cytoplasmic calcium ions in the cell in which the Caᵥ1.2 channel is present by at least about 5% or more, such as, but not limited to, about 10%, 20%, 30%, 40%, 50%, 75%, 100%, 150%, 200%, 300%, or more.

In certain embodiments, a method of the present invention increases the rate of calcium ion influx into the cell in which the Caᵥ1.2 channel is present compared to the rate of calcium ion influx into the cell or a comparable cell in the absence of a method of the present invention. A method of the present invention may increase the rate of calcium ion influx into the cell in which the Caᵥ1.2 channel is present by at least about 5% or more, such as, but not limited to, about 10%, 20%, 30%, 40%, 50%, 75%, 100%, 150%, 200%, 300%, or more. A method of the present invention may slow the closing of the Caᵥ1.2 channel. This may allow for more calcium ions to influx into the cell in which the Caᵥ1.2 channel is present. As those skilled in the art will recognize, modulating a Caᵥ1.2 channel may cause one or more Caᵥ1.2 channel activities to increase and may cause one or more Caᵥ1.2 channel activities to decrease. For example, a method of the present invention may increase the transport of calcium ions (e.g., Ca²⁺) into the cell in which the Caᵥ1.2 channel is present, but may decrease the rate of closing of the Caᵥ1.2 channel.

In some embodiments, a method of the present invention increases the ability of the cell in which the Caᵥ1.2 channel is present to generate and/or transmit electrical signals compared to the cell's ability or a comparable cell's ability to generate and/or transmit electrical signals in the absence of a method of the present invention. A method of the present invention may increase the ability of the cell in which the Caᵥ1.2 channel is present to generate and/or transmit electrical signals by at least about 5% or more, such as, but not limited to, about 10%, 20%, 30%, 40%, 50%, 75%, 100%, 150%, 200%, 300%, or more.

In some embodiments, a method of the present invention may decrease the rate of Caᵥ1.2 channel inactivation and/or the amount of time it takes for cellular repolarization compared to the rate of Cᵥ1.2 channel inactivation and/or the amount of time it takes for cellular repolarization in the cell or a comparable cell in the absence of a method of the present invention. A method of the present invention may decrease the rate of Caᵥ1.2 channel inactivation and/or the amount of time it takes for cellular repolarization by at least about 5% or more, such as, but not limited to, about 10%, 20%, 30%, 40%, 50%, 75%, 80%, 85%, 90%, 95%, 97%, or more.

According to some embodiments, a method of increasing bone mass in a subject is provided comprising modulating a Caᵥ1.2 channel activity in bone of the subject, thereby increasing bone mass in the subject. “Bone mass,” “bone density,” and “bone mineral density” are used interchangeably herein and refer to the concentration of one or more minerals (e.g., calcium, phosphorous, magnesium, etc.) in a particular section of a bone or a bone volume. Bone mass, bone density, and/or bone mineral density may be measured using methods known in those skilled in the art, such as, but not limited to, dual-energy x-ray absorptiometry (DXA). A method of the present invention may increase bone mass and/or may increase the strength of the bone.

Alternatively or in addition, a method of preventing or slowing bone mineral density loss in bone of a subject may be provided, the method comprising modulating Caᵥ1.2 channel activity in bone of the subject, thereby preventing or slowing bone mineral density loss in bone of the subject. In some embodiments, a method of the present invention prevents or slows the loss of a mineral, such as, but not limited to, calcium, phosphorous, magnesium, and any combination thereof In certain embodiments, a method of the present invention reverses bone loss that has occurred in a subject.

According to some embodiments, a method of preventing and/or treating osteoporosis in a subject is provided, the method comprising modulating Caᵥ1.2 channel activity in bone of the subject, thereby preventing and/or treating osteoporosis in the subject. Alternatively or in addition, a method of preventing the occurrence of compression fractures due to osteoporosis may be provided, the method comprising modulating Caᵥ1.2 channel activity in bone of the subject, thereby preventing the occurrence of compression fractures due to osteoporosis. A “compression fracture,” as used herein, is a broken, cracked and/or collapsed vertebra. In some embodiments, a method of the present invention comprises increasing the calcified bone fraction in the bone. In some embodiments, a method of the present invention comprises treating a traumatic bone injury (i.e., a bone fracture due to trauma), the method comprising modulating Caᵥ1.2 channel activity in the injured bone of the subject, thereby treating the traumatic bone injury. A method of the present invention may increase bone density and/or bone strength.

The terms “prevent,” “preventing” and “prevention of” (and grammatical variations thereof) refer to reduction and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

By the term “treat,” “treating” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder.

A “treatment effective” amount as used herein is an amount that is sufficient to treat (as defined herein) the subject. Those skilled in the art will appreciate that the therapeutic effects need not be complete or curative, as long as some benefit is provided to the subject.

The present invention finds use in both veterinary and medical applications. Suitable subjects of the present invention include, but are not limited to avians and mammals. The term “avian” as used herein includes, but is not limited to, chickens, ducks, geese, quail, turkeys, pheasants, parrots, parakeets, macaws, cockatiels, canaries, and finches. The term “mammal” as used herein includes, but is not limited to, primates (e.g., simians and humans), non-human primates (e.g., monkeys, baboons, chimpanzees, gorillas), bovines, ovines, caprines, ungulates, porcines, equines, felines, canines, lagomorphs, pinnipeds, rodents (e.g., rats, hamsters, and mice), etc. In some embodiments of the present invention the subject is a mammal and in certain embodiments the subject is a human. Human subjects include both males and females and subjects of all ages including fetal, neonatal, infant, juvenile, adolescent, adult, and geriatric subjects.

In particular embodiments, the subject is “in need of” or “in need thereof” a method of the present invention, for example, the subject is in an at-risk population (e.g. the subject may be at-risk for or more susceptible to a bone fracture or osteoporosis) and/or the subject has a disorder and/or condition that may be treated with a method of the present invention. The present invention may be particularly suitable for geriatric subjects and postmenopausal women. In some embodiments, the subject has osteoporosis, a bone fracture, failed arthrodesis, dyschondroplasia, achondroplasia, congenital pseudoarthrosis, a stress fracture, and any combination thereof. In some embodiments, the subject has an increased risk for bone stress. In some embodiments, the subject (e.g., a soldier, athlete, etc.) has an increased risk of a bone injury. In certain embodiments, the subject has a bone injury, such as a traumatic bone injury. In certain embodiments, a method of the present invention minimizes cardiovascular side effects or has no cardiovascular side effects.

According to some embodiments, modulating a Caᵥ1.2 channel activity may be carried out by administering at least one active agent to the subject, wherein the active agent is a Caᵥ1.2 channel activator. A “Caᵥ1.2 channel activator,” as used herein, refers to a compound that modulates a Caᵥ1.2 channel. A Caᵥ1.2 channel activator may increase a Caᵥ1.2 channel activity.

In certain embodiments, a Caᵥ1.2 channel activator increases the activity of an endogenous Caᵥ1.2 channel. A Caᵥ1.2 channel activator may be specific for bone and/or osteoblasts and/or may activate bone formation and/or osteoblasts. In some embodiments, a method of the present invention may increase the concentration of at least one serum marker of osteoblasts and/or bone formation (e.g., osteoprotegerin and osteocalcin).

In some embodiments, a Caᵥ1.2 channel activator mimics a gain-of-function mutant L-type (Caᵥ1.2) voltage-gated Ca²⁺ channel (i.e., a gain-of-function Caᵥ1.2 mutant). An exemplary gain-of-function Caᵥ1.2 mutant is the Caᵥ1.2 (G406R) mutant which is a result of a gain-of-function mutation (G406R) in CACNA1C as described in Ramachandran et al., “Calcium influx through L-type Caᵥ1.2 Ca²⁺ channels regulates mandibular development” The Journal of Clinical Investigation, 2013; (123)(4), which is incorporated herein by reference in its entirety. The Caᵥ1.2 channel activator may mimic an action, behavior, and/or response of a gain-of-function Caᵥ1.2 mutant when the gain-of-function Caᵥ1.2 mutant is expressed or over-expressed in a cell.

A Caᵥ1.2 channel activator may comprise a small organic compound. A “small organic compound,” as used herein, refers to an organic compound having a molecular weight of more than about 10 Daltons and less than about 5,000 Daltons, or any range therein, such as from about 40 Daltons to about 3,000 Daltons, from about 100 Daltons to about 2,500 Daltons, or from about 100 Daltons to about 1,000 Daltons. A small organic compound can be natural, modified, or synthetic. Small organic compounds of the present invention can comprise functional groups necessary for structural interaction with bones. Exemplary small organic compounds include, but are not limited to, pharmaceuticals, sugars, fatty acids, steroids, saccharides, purines, pyrimidines, derivatives, structural analogs, or combinations thereof.

In some embodiments, a Caᵥ1.2 channel activator may comprise at least one channel agonist. Exemplary Caᵥ1.2 channel activators include, but are not limited to, (±)-Bay K8644 (1,4-Dihydro-2,6-dimethyl-5-nitro-4-(2-[trifluoromethyl]phenyl)pyridine-3-carboxylic acid methyl ester), FPL 64176 (2,5-Dimethyl-4-[2-(phenylmethyl)benzoyl]-1H-pyrrole-3-carboxlic acid methyl ester), and any combination thereof.

According to some embodiments, a Caᵥ1.2 channel activator may comprise means for targeting bone. Exemplary means for targeting bone include, but are not limited to, tetracycline, a tetracycline derivative, a bisphosphonate, D-aspartic acid octapeptide (D-Asp₈), and any combination thereof. Further exemplary means for targeting bone include those described in Neale et al. Bioorganic & Medicinal Chemistry Letters, 19 (2009) 680-683 and Wang et al. Mol. Pharm. (2006); 3(6): 717-725, each of which are incorporated by reference herein for the portions relevant to this paragraph. A Caᵥ1.2 channel activator may be conjugated to said means for targeting bone using methods known to those of skill in the art.

An active agent may be administered to a subject using any suitable means to carry out an embodiment of the present invention. Exemplary modes of administration include, but are not limited to, administration by ingestion via the oral route, intranasal, rectal, inhalation, topical, transdermal, or injection, such as intravenous, subcutaneous, intramuscular, intraperitoneal, intracranial, and spinal injection.

The active agents disclosed herein may be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

Active agents may be administered as prodrugs. “Prodrugs” as used herein refers to those prodrugs of the compounds of the present invention which are, within the scope of sound medical judgment, suitable for use in contact with the tissues of humans and lower animals without undue toxicity, irritation, allergic response and the like, commensurate with a reasonable risk/benefit ratio, and effective for their intended use, as well as the zwitterionic forms, where possible, of the compounds of the invention. The term “prodrug” refers to compounds that are rapidly transformed in vivo to yield the parent compound of the above formulae, for example, by hydrolysis in blood. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299. Examples include a prodrug that is metabolized in vivo by a subject to an active drug having an activity of active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. No. 6,680,324 and U.S. Pat. No. 6,680,322.

The active agents described above may be formulated for administration in a pharmaceutical carrier in accordance with known techniques. See, e.g., Remington, The Science And Practice of Pharmacy (9th Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy comprising admixing the components, optionally including one or more accessory ingredients.

The formulations of the invention include those suitable for oral, topical, buccal (e.g., sub-lingual), vaginal, rectal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy that includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the active compound(s), which preparations are preferably isotonic with the blood of the intended recipient. These preparations may contain anti-oxidants, buffers, bacteriostats and solutes that render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active agent(s), or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent that is physiologically acceptable may be employed in sufficient quantity to emulsify the compound or salt in an aqueous carrier. One such useful emulsifying agent is phosphatidyl choline.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis/tris buffer (pH 6) or ethanol/water and contain from 0.1 to 0.2 M active ingredient.

In addition to active compound(s), the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

The therapeutically effective dosage of any specific active agent, the use of which is in the scope of present invention, will vary somewhat from compound to compound, and patient to patient, and will depend upon the condition of the patient and the route of delivery. For oral administration, a total daily dosage of about 10 mg to about 10 g may be used, given as a single daily dose or divided into two or three daily doses.

Instructions for use may be packaged with or otherwise associated with an active agent indicating recommendations for treatment, time to treatment, dose regimens, etc., based upon the presence or absence of the genetic variants.

In some embodiments, an active agent may be administered concurrently or sequentially with an additional therapeutic agent, such as, but not limited to a dihydropyridine calcium channel blocker.

The present invention is explained in greater detail in the following non-limiting Examples.

### EXAMPLES

### Example 1

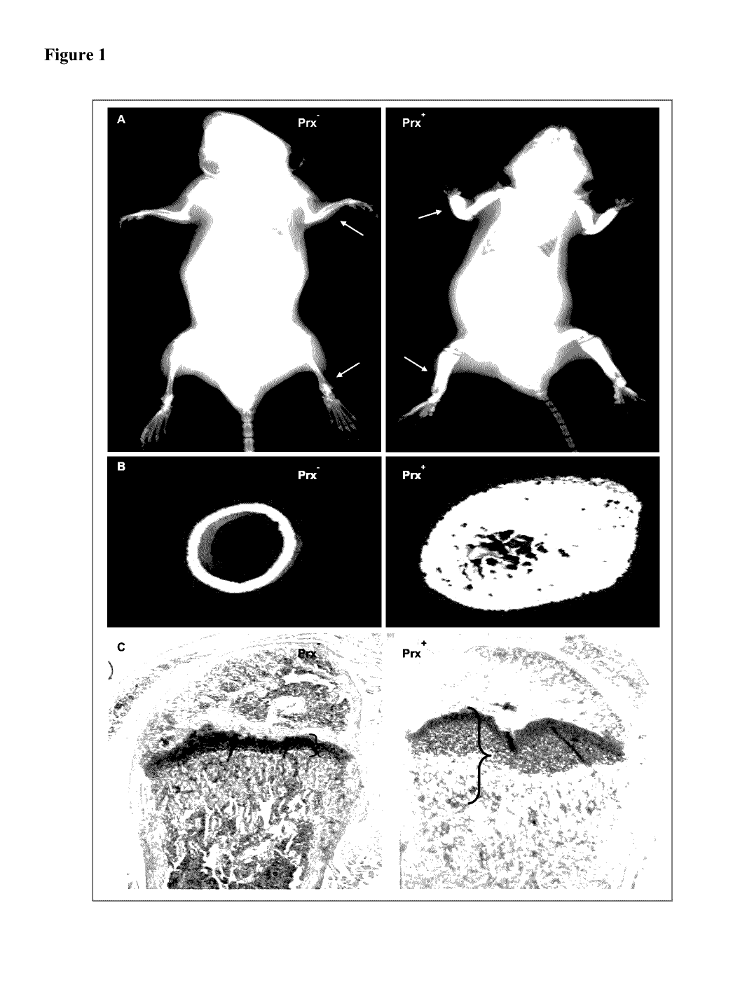

It was discovered that targeted expression of a gain-of-function mutant Caᵥ1.2 in bone leads to a massive increase in bone density. Using a Cre-based strategy, a gain-of-function mutant Caᵥ1.2 (Caᵥ1.2^{TS}) restricted to limb bud mesenchyme was expressed and observed a massive increase in bone density (FIG. 1, Panel A). Equivalent expression of a wild-type Caᵥ1.2 (Caᵥ1.2^{WT}) had no effect. The mutant Caᵥ1.2 (G406R) was originally discovered as the cause for Timothy Syndrome (TS), a rare disorder characterized by life-threatening cardiac arrhythmias and syndactyly². The mutation greatly slows channel closing, resulting in excessive Ca²⁺ influx. Tissue-restricted expression was achieved by exploiting a mouse model in which a silenced (by an upstream floxed STOP codon) Caᵥ1.2^{TS} or Caᵥ1.2^{WT} (knocked into the Rosa26 locus)³ was activated by Prxl-Cre. Prxl drives expression in the developing limb bud mesenchyme⁴. As seen in FIG. 1, Panel A (right), a radiograph shows markedly increased density in the appendicular, but not the axial, skeleton of a Prxl⁺-Caᵥ1.2^{TS} mouse, consistent with the Prxl-delimited expression pattern. A radiograph of a Prxl⁻ littermate, in contrast, revealed no increase in bone density in either the appendicular or the axial skeleton (FIG. 1, Panel A, left). Quantitative assessment of bone mineral density by dual-energy x-ray absorptiometry (DXA) showed a greater than 2-fold increase in the femur of Prxl⁺-Caᵥ1.2^{TS} mice vs. Prxl⁻ littermates (0.11±0.003 vs. 0.05±0.002 g/cm², p<10⁻⁶, N=3-6). A μCT of the mid-femur shafts was obtained, which revealed a marked increase in overall bone thickness and osteopetrosis in Prxl⁺-Caᵥ1.2^{TS} mice (FIG. 1, Panel B). Serum markers of osteoblasts (bone building cells) and bone formation (osteoprotegerin and osteocalcin, respectively) were elevated greater than 2-fold in Prxl⁺-Caᵥ1.2^{TS} mice compared to their Prxl⁻ littermates. It was confirmed that the increased bone density was due to expression of the gain-of-function Caᵥ1.2^{TS} mutant channel, and not simply to over-expression of Caᵥ1.2: measurements in Prxl⁺-Caᵥ1.2^{WT}and Prxl⁻-Caᵥ1.2^{WT} mice were not different from each other or from a wild type mouse (not shown). Safranin O staining of distal femur tissue sections for Prxl-Caᵥ1.2^{TS} mice showed a marked increase of the proliferative and hypertrophic chondrocyte zones (brackets) within the growth plate cartilage in Prxl⁺ mice, as well as increased trabecular bone, which is formed by osteoblasts (FIG. 1, Panel C).

While not wishing to be bound to any particular theory, since Prxl directs expression in osteoblasts and chondrocytes, but not in the bone-resorbing osteoclasts⁵, these data may provide clues to possible cellular mechanisms by which targeted expression of Caᵥ1.2^{TS} increases bone mass. One hypothesis is that Caᵥ1.2^{TS} in chondrocytes leads to increased levels of secreted factors known to attract invasion of blood vessels, which osteoblasts then follow to replace a skeletal structure left behind by apoptotic chondrocytes. Alternatively, Caᵥ1.2 expression in osteoblasts may increase osteoblastic activity or change the balance between osteoblasts and osteoclasts. In support of this second hypothesis is the increased serum osteoprotegerin in the Prxl-Caᵥ1.2^{TS} mice (see above). Osteoblast-secreted osteoprotegerin binds to, and masks RANKL, a surface-bound molecule on osteoblasts necessary for activating osteoclasts. Masking of RANKL by increased osteoprotegerin in the Prxl-Caᵥ1.2^{TS} mice should reduce the number of bone-resorbing osteoclasts and change the balance with osteoblasts. Indeed, Prxl-Cre driven deletion of RANKL leads to an expansion of the growth plate cartilage⁵, similar to what we observed in our model (FIG. 1, Panel C). Thus, the increase in bone mass may result from either an osteoblast and/or chondrocyte mechanism.

### Example 2

Experiments will be performed to identify dihydropyridine agonists that are able to activate endogenous Caᵥ1.2 channels in vivo in a manner similar to the Caᵥ1.2^{TS} mutants. Tamoxifen-inducible Col1a-Cre and Col2a-Cre lines (from JAX) have been obtained and will be crossed with a STOP-floxed Caᵥ1.2^{TS} line. Female offspring carrying a Cre (choice of a Col1a-Cre or Col2a-Cre line) and the STOP-floxed Caᵥ1.2^{TS} allele will be aged to 10 weeks. The mice will then undergo ovariectomy, which induces an almost immediately measurable loss in bone density, and is an established model for osteoporosis. Ovariectomized mice will be treated with tamoxifen to induce targeted Caᵥ1.2^{TS} expression and bone density will be analyzed 6 weeks later. An increase in bone mass or any reversal of the expected decrease of bone mass (compared to controls that do not receive tamoxifen) will indicate that activation of mutant Caᵥ1.2 channels in adult bone could be a viable strategy for osteoporosis therapy.

### Example 3

Experiments will be performed in which oviarectomized mice will be treated with a Caᵥ1.2 channel activator that is targeted to bone. Different means for targeting the Caᵥ1.2 channel activator to bone will be evaluated and suitable dosages of the Caᵥ1.2 channel activator will be determined.

### Example 4

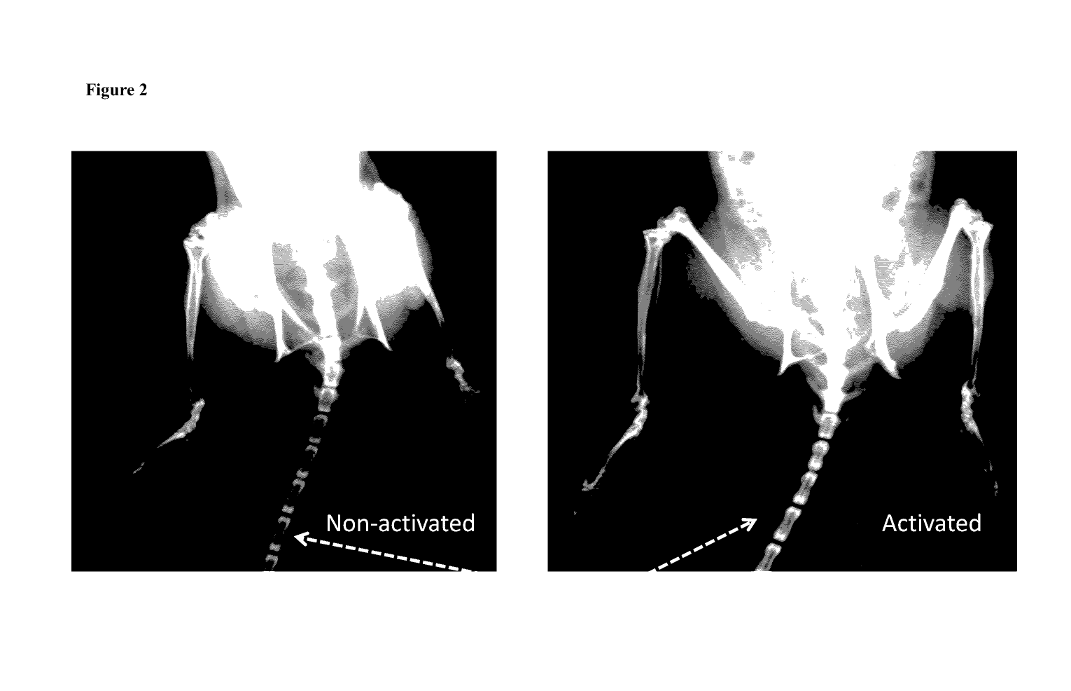

Using a separate Cre based strategy in which the Caᵥ1.2^{TS} channel was targeted to bone (entire skeleton via a Col1a1-Cre) in a manner in which expression could be induced at will, the channel was activated in adult mice (age 8 weeks) and bone density was analyzed by x-radiography at 5 months of age. As shown in FIG. 2, bone density increased in animals with the activated channel (most clearly observed in the tail vertebrae in which less soft tissue obscures the signal). This demonstrates that activation of the Caᵥ1.2^{TS} channel in adult animals, which can be mimicked by pharmacological agents, increases bone density and thereby provides a basis for treating adult subjects.

The foregoing is illustrative of the present invention, and is not to be construed as limiting thereof. The invention is defined by the following claims, with equivalents of the claims to be included therein. All publications, patent applications, patents, patent publications, and other references cited herein are incorporated by reference in their entireties for the teachings relevant to the sentence and/or paragraph in which the reference is presented.

### REFERENCES

1. Sambrook and Cooper “Osteoporosis” Lancet 367:2010-2018 (2006)

2. Splawski et al. “Ca(V)1.2 calcium channel dysfunction causes a multisystem disorder including arrhythmia and autism” Cell 119:19-31 (2004)

3. Pasca et al. “Using iPSC-derived neurons to uncover cellular phenotypes associated with Timothy syndrome” Nat Med 17:1657-1662 (2011)

4. Logan et al. “Expression of Cre Recombinase in the developing mouse limb bud driven by a Prxl enhancer” Genesis 33:77-80 (2002)

5. Xiong et al. “Matrix-embedded cells control osteoclast formation” Nat Med 17:1235-1241 (2011)

6. Guggino et al. “Phenylalkylamine-sensitive calcium channels in osteoblast-like osteosarcoma cells. Characterization by ligand binding and single channel recordings” J Biol Chem 263:10155-10161 (1988)

7. Gu et al. “Osteoblasts derived from load-bearing bones of the rat express both L- and T-like voltage-operated calcium channels and mRNA for a1C , a1D and a1G subunits” Pflugers Archiv European Journal of Physiology 438:553-560 (1999)

8. Guggino et al. “Bone remodeling signaled by a dihydropyridine- and phenylalkylamine-sensitive calcium channel” PNAS 86:2957-2960 (1989)

9. Shao et al. “Expression of voltage sensitive calcium channel (VSCC) L-type Cav1.2 (alpha1C) and T-type Cav3.2 (alpha1H) subunits during mouse bone development” Dev Dyn 234:54-62 (2005)

10. Mancilla et al. “L-type calcium channels in growth plate chondrocytes participate in endochondral ossification” J Cell Biochem 101:389-398 (2007)

11. Seisenberger et al. “Functional Embryonic Cardiomyocytes after Disruption of the L-type a1C (Cav1.2) Calcium Channel Gene in the Mouse” J. Biol. Chem. 275:39193-39199 (2000)

12. Rosati et al. “Robust L-type calcium current expression following heterozygous knockout of the Cav1.2 gene in adult mouse heart” J Physiol 589:3275-3288 (2011)

13. Tomita et al. Calcineurin and NFAT4 induce chondrogenesis. J Biol Chem 277:42214-42218 (2002)

That which is claimed is:

1. A method of increasing bone mass in a subject, comprising modulating Caᵥ1.2 channel activity in bone of the subject, thereby increasing bone mass in the subject.

2. A method of preventing and/or treating osteoporosis and/or preventing the occurrence of compression fractures due to osteoporosis in a subject in need thereof, comprising modulating Caᵥ1.2 channel activity in bone of the subject, thereby preventing and/or treating osteoporosis and/or preventing the occurrence of compression fractures due to osteoporosis in the subject.

3. A method of preventing or slowing bone mineral density loss in bone of a subject in need thereof, comprising modulating Caᵥ1.2 channel activity in bone of the subject, thereby preventing or slowing bone mineral density loss in bone of the subject.

4. The method of claim 1, wherein said step of modulating Caᵥ1.2 channel activity is carried out by administering to the subject a Caᵥ1.2 channel activator.

5. The method of claim 4, wherein said Caᵥ1.2 channel activator is bone specific and/or osteoblast specific.

6. The method of claim 4, wherein said Caᵥ1.2 channel activator mimics a gain-of-function mutant L-type (Caᵥ1.2) voltage-gated Ca²⁺ channel.

7. The method of claim 4, wherein said Caᵥ1.2 channel activator comprises at least one channel agonist.

8. The method of claim 7, wherein said at least one channel agonist is at least one of (±)-Bay K8644 (1,4-Dihydro-2,6-dimethyl-5-nitro-4-(2-[trifluoromethyl]phenyl)pyridine-3-carboxylic acid methyl ester) and FPL 64176 (2,5-Dimethyl-4-[2-(phenylmethyl)benzoyl]-1H-pyrrole-3-carboxlic acid methyl ester).

9. The method of claim 4, wherein said Caᵥ1.2 channel activator comprises means for targeting bone.

10. The method of claim 9, wherein said means for targeting bone comprises a tetracycline, a tetracycline derivative, a bisphosphonate, D-aspartic acid octapeptide (D-Asp₈), and any combination thereof.

11. The method of claim 1, further comprising administering to said subject a dihydropyridine calcium channel blocker.

12. The method of claim 1, wherein said step of modulating Caᵥ1.2 channel activity is configured to slow closing of said channel.

13. The method of claim 1, wherein said step of modulating Caᵥ1.2 channel activity is configured to increase influx of calcium ions into the cell in which said channel is present.

14. The method of claim 1, wherein said step of modulating Caᵥ1.2 channel activity is configured to decrease the rate of Caᵥ1.2 channel inactivation.

15. The method of claim 1, wherein said step of modulating Caᵥ1.2 channel activity is configured to increase the concentration of at least one serum marker of osteoblasts and bone formation.

16. The method of claim 1, wherein said step of modulating Caᵥ1.2 channel activity is carried out by at least one of an osteoblast-specific mechanism and a chondrocyte-specific mechanism.

17. The method of claim 1, wherein said method further comprises increasing the calcified bone fraction in said bone.

18. The method of claim 1, wherein said subject has osteoporosis, bone fracture, failed arthrodesis, dyschondroplasia, achondroplasia, congenital pseudoarthrosis and/or a stress fracture.

19. The method of claim 1, wherein said subject has an increased risk for bone stress.

20. The method of claim 1, wherein said method minimizes cardiovascular side effects or has no cardiovascular side effects.

21. A compound comprising a Caᵥ1.2 channel activator that increases a Caᵥ1.2 channel activity.

22. The compound of claim 21, wherein said Caᵥ1.2 channel activator activates at least one of bone formation and osteoblasts.

23. The compound of claim 21, wherein said Caᵥ1.2 channel activator mimics a gain-of-function mutant L-type (Caᵥ1.2) voltage-gated Ca²⁺ channel.

24. The compound of claim 21, wherein said Caᵥ1.2 channel activator comprises at least one channel agonist.

25. The compound of claim 24, wherein said at least one channel agonist is at least one of (±)-Bay K8644 (1,4-Dihydro-2,6-dimethyl-5-nitro-4-(2-[trifluoromethyl]phenyl)pyridine-3-carboxylic acid methyl ester) and FPL 64176 (2,5-Dimethyl-4-[2-(phenylmethyl)benzoyl]-1H-pyrrole-3-carboxlic acid methyl ester).

26. The compound of claim 21, wherein said Caᵥ1.2 channel activator comprises means for targeting bone.

27. The compound of claim 26, wherein said means for targeting bone comprises tetracycline, a tetracycline derivative, a bisphosphonate, D-aspartic acid octapeptide (D-Asp₈), and any combination thereof.
